(19) United States
(12) Patent Application Publication
(10) Pub. No.: US 2026/0074077 A1
(43) Pub. Date: Mar. 12, 2026

(54) PATIENT DECISION SUPPORT SYSTEMS AND METHODS UTILIZING AUGMENTED REALITY AND MACHINE LEARNING

(71) Applicant: Mission Critical Protocols, Inc., San Diego, CA (US)

(72) Inventors: John Ehrhart, San Diego, CA (US); Natanael de Kross, Charlotte, NC (US); Matthew Penning, San Diego, CA (US)

(73) Assignee: Mission Critical Protocols, Inc., San Diego, CA (US)

(21) Appl. No.: 19/325,084

(22) Filed: Sep. 10, 2025

Related U.S. Application Data
(60) Provisional application No. 63/692,830, filed on Sep. 10, 2024.

(51) Int. Cl. G16H 50/30; A61B 5/00; A61B 5/107; A61B 90/00; A61M 16/00; G16H 40/67

(52) U.S. Cl. CPC G16H 50/30; A61B 5/0077; A61B 5/1072; A61B 5/1079; A61B 5/7267; A61B 5/7278; G16H 40/67; A61B 2090/365; A61B 2505/01; A61M 16/024

(57) ABSTRACT

Software systems utilizing augmented reality (such as via a smartphone) allow for improved accuracy of patient body mass measurement in emergency medicine situations, improving patient outcomes and speeding administration of lifesaving care.

### CROSS-REFERENCE TO RELATED APPLICATION

This application claims priority to and the benefit of U.S. Provisional Application No. 63/692,830 filed Sep. 10, 2024, entitled “PATIENT DECISION SUPPORT SYSTEMS AND METHODS UTILIZING AUGMENTED REALITY AND MACHINE LEARNING.” The foregoing application is hereby incorporated by reference in its entirety, including but not limited to those portions that specifically appear hereinafter, but except for any subject matter disclaimers or disavowals, and except to the extent that the incorporated material is inconsistent with the express disclosure herein, in which case the language in this disclosure shall control.

### TECHNICAL FIELD

The present disclosure relates to augmented reality (AR), and particularly to use of augmented reality and machine learning (ML) in connection with emergency medical services.

### BACKGROUND

Emergency Medical Services (EMS) operate in high-stress environments where quick, accurate decision-making is critical to patient outcomes. One of the challenges EMS providers face is obtaining accurate patient measurements, such as height and weight, which are crucial for determining appropriate medication dosages, fluid resuscitation, ventilator settings, bone fracture management, equipment sizing, defibrillation, and more. Traditional methods, such as length-based resuscitation tapes for pediatric patients and adult estimation techniques, can be imprecise and time-consuming. These methods also introduce variability in treatment, which can impact patient safety and care outcomes. Accordingly, improved approaches remain desirable.

### SUMMARY

In an exemplary embodiment, a computerized method for determining and utilizing patient metrics in an emergency medicine context via use of augmented reality (AR) and machine learning (ML) comprises: obtaining, via a camera system of a mobile device, information regarding the height of a patient, wherein the obtaining utilizes augmented reality capabilities of the camera system; determining, by a machine learning system operative on the mobile device, an estimated weight of the patient, and recommending, by a decision support system operative on the mobile device, treatment parameters for the patient based on the determining.

The machine learning may utilize a deep learning based neural network. The camera system may comprise light detection and ranging (LiDAR) capability, and the obtaining information regarding the height of a patient may utilize the camera system. The camera system may lack LiDAR capability. The treatment may comprise administering a medication, and the treatment parameters may comprise dosage information based on the weight of the patient. The treatment may comprise utilizing a medical device, and the treatment parameters may comprise sizing information for the medical device based on the weight of the patient.

The treatment may comprise ventilating the patient, and the treatment parameters may comprise ventilator configuration information based on the weight of the patient. The ventilator configuration information may comprise tidal volume and positive end-expiratory pressure (PEEP). The determining the estimated weight of the patient may comprise generating a mesh representative of the body of the patient. The recommending may comprise displaying, on a graphical user interface of the mobile device, treatment parameters comprising dosage information based on the weight of the patient. The method may further comprise administering, to the patient, medication corresponding to the dosage information. The method may further comprise transmitting, to a patient treatment database and by the mobile device, a record of the administered medication. The method may further comprising inputting, to a patient treatment database and by the mobile device, a record of the administered medication.

The foregoing are intended as a simplified introduction to the disclosure, and are not intended to limit the scope of any claim.

### BRIEF DESCRIPTION OF THE DRAWINGS

With reference to the following description and accompanying drawings:

FIG. 1A illustrates a block diagram of an exemplary patient metrics system, in accordance with various exemplary embodiment;

FIG. 1B illustrates a screenshot of an exemplary patient metrics software application, in accordance with various exemplary embodiments;

FIG. 2 illustrates a screenshot of an exemplary patient metrics software application prior to selections or measurements, in accordance with various exemplary embodiments;

FIG. 3 illustrates a screenshot of an exemplary patient metrics software application whereby age is selected or auto-populated, in accordance with various exemplary embodiments;

FIG. 4 illustrates a screenshot of an exemplary patient metrics software application wherein biological sex is selected, in accordance with various exemplary embodiments;

FIG. 5 illustrates a screenshot of an exemplary patient metrics software application showing scanning of an environment, in accordance with various exemplary embodiments;

FIG. 6 illustrates a screenshot of an exemplary patient metrics software application showing marking of a patient head, in accordance with various exemplary embodiments;

FIG. 7 illustrates a screenshot of an exemplary patient metrics software application showing marking of a patient's heels, in accordance with various exemplary embodiments;

FIG. 8 illustrates a screenshot of an exemplary patient metrics software application showing certain patient calculations and other displayed information, in accordance with various exemplary embodiments;

FIG. 9 illustrates a screenshot of an exemplary patient metrics software application showing confirmation of absolute body weight, in accordance with various exemplary embodiments;

FIG. 10 illustrates a screenshot of an exemplary patient metrics software application showing manual adjustment of absolute body weight, in accordance with various exemplary embodiments;

FIG. 11 illustrates a screenshot of an exemplary patient metrics software application showing acceptance of absolute body weight for use as a driver of additional calculations, in accordance with various exemplary embodiments;

FIG. 12 illustrates a screenshot of an exemplary patient metrics software application showing management of patients and metrics, in accordance with various exemplary embodiments;

FIGS. 13 and 14 illustrate screenshots of an exemplary patient metrics software application showing selection of medication and calculation of dosing and volume guidance, in accordance with various exemplary embodiments;

FIG. 15 illustrates a screenshot of an exemplary patient metrics software application showing selection of equipment based on patient characteristics, in accordance with various exemplary embodiments; and

FIG. 16 illustrates a flowchart showing use and functions of an exemplary patient metrics software application in accordance with an exemplary method 1600, in accordance with various exemplary embodiments.

### DETAILED DESCRIPTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from the scope of the present disclosure.

The detailed description of various embodiments herein refer to the accompanying drawings and pictures, which show various embodiments by way of illustration. While these various embodiments are described in sufficient detail to enable those skilled in the art to practice the disclosure, it should be understood that other embodiments may be realized, and that logical, communicative, functional, and/or similar changes may be made without departing from the spirit and scope of the disclosure. Thus, the detailed description herein is presented for purposes of illustration only and not of limitation. For example, the steps recited in any of the method or process descriptions may be executed in any suitable order and are not limited to the order presented. Moreover, certain of the functions or steps may be outsourced to or performed by one or more third parties. Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used herein, “each” refers to each member of a set or each member of a subset of a set. Furthermore, any reference to singular includes plural embodiments, and any reference to more than one component may include a singular embodiment. Although specific advantages have been enumerated herein, various embodiments may include some, none, or all the enumerated advantages.

Exemplary principles of the present disclosure contemplate use of AR-enhanced systems, machine learning, and/or artificial intelligence in connection with healthcare decision making, for example patient measurement. In an exemplary embodiment as represented by a patient metrics software application operative on a smartphone, an AR component measures the patient, generating height and ideal body weight. The system then calculates absolute body weight using a deep learning-based neural network. These patient measurements are automatically used to calculate medication doses and ventilator settings needed during emergency care, replacing traditional length-based resuscitation tapes for pediatrics and estimation methods for adults. By automating these critical calculations, principles of the present disclosure improve precision and efficiency in emergency situations, reducing cognitive load on EMS providers and enhancing patient outcomes.

Exemplary embodiments improve the accuracy and efficiency of patient measurements in the EMS setting by integrating augmented reality (AR) and deep learning technologies. The AR component allows EMS providers to quickly measure patient height while the deep learning algorithms calculate ideal and absolute body weights. These measurements can then be used directly through integrated tools that rely on such measurements to provide effective and accurate care to the patient.

### Exemplary Embodiments Solve the Following Problems:

Inaccurate Patient Measurements: Traditional methods of estimating patient height and weight are prone to error, leading to potential inaccuracies in medication dosing and ventilator settings. Exemplary embodiments provide a more accurate and reliable method for obtaining these measurements, enhancing the precision of emergency care.

Time-Consuming Processes: Time is critical in emergency scenarios. Traditional methods of measuring or estimating patient metrics can be slow and cumbersome, delaying essential care. Exemplary embodiments streamline the process by allowing for rapid and automated measurements, saving valuable time during emergency response.

Variability in Care: Different EMS providers may use varying techniques to estimate patient metrics, leading to inconsistencies in care. Exemplary embodiments reduce variability by standardizing the measurement process through AR and deep learning, ensuring that all patients receive care based on accurate, consistent data.

Limited Applicability of Existing Tools: Existing tools, such as length-based resuscitation tapes, are primarily designed for pediatric patients and only provide approximations, making them less accurate and generally inapplicable to adult patients. Mechanical or digital scales, essential for accurate weight measurement, are often inaccessible or impractical to use in many emergencies, particularly in the field.

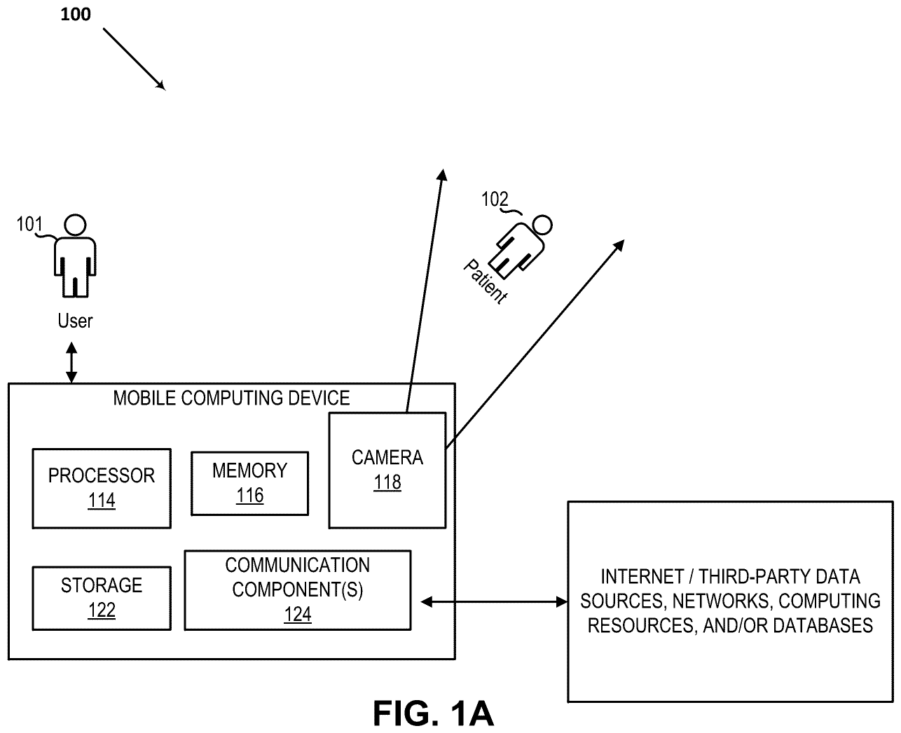

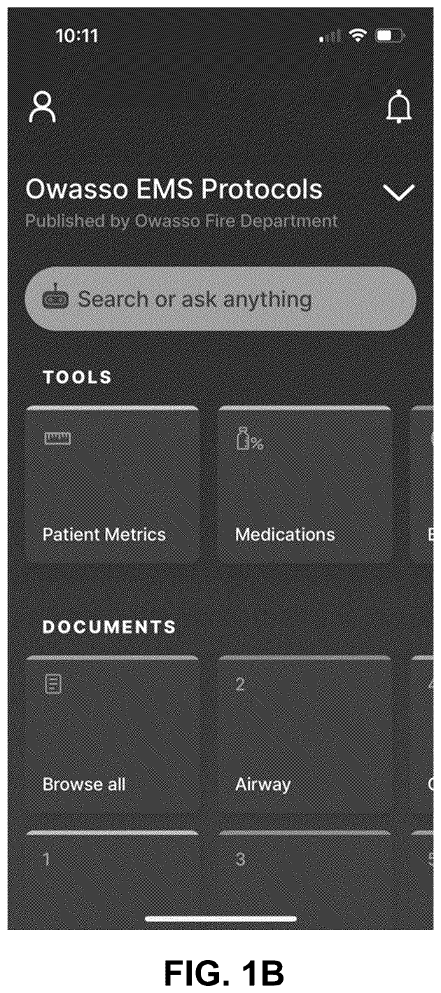

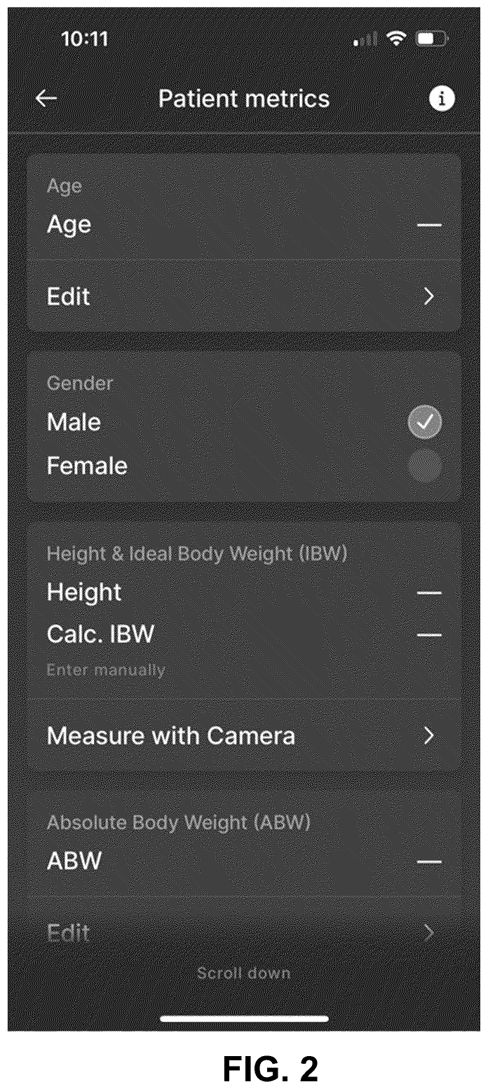

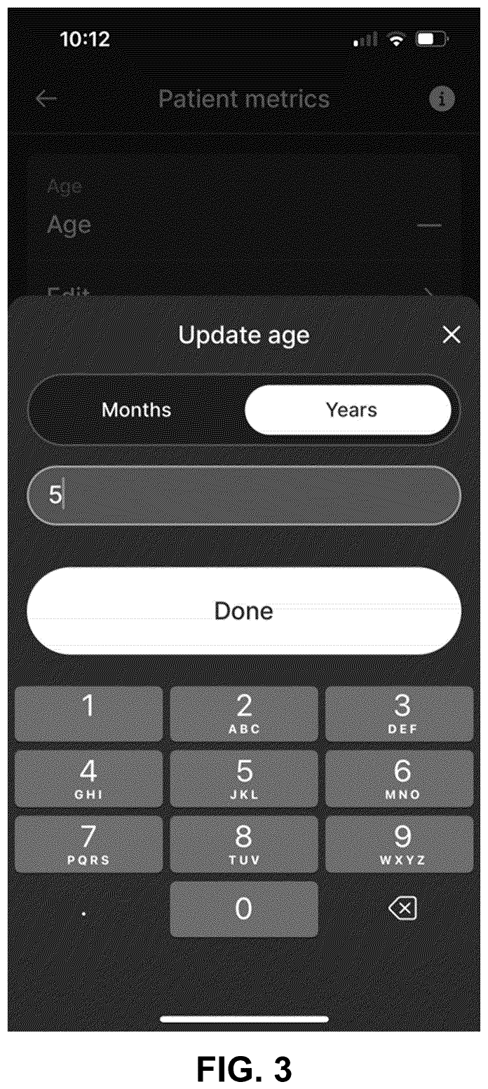

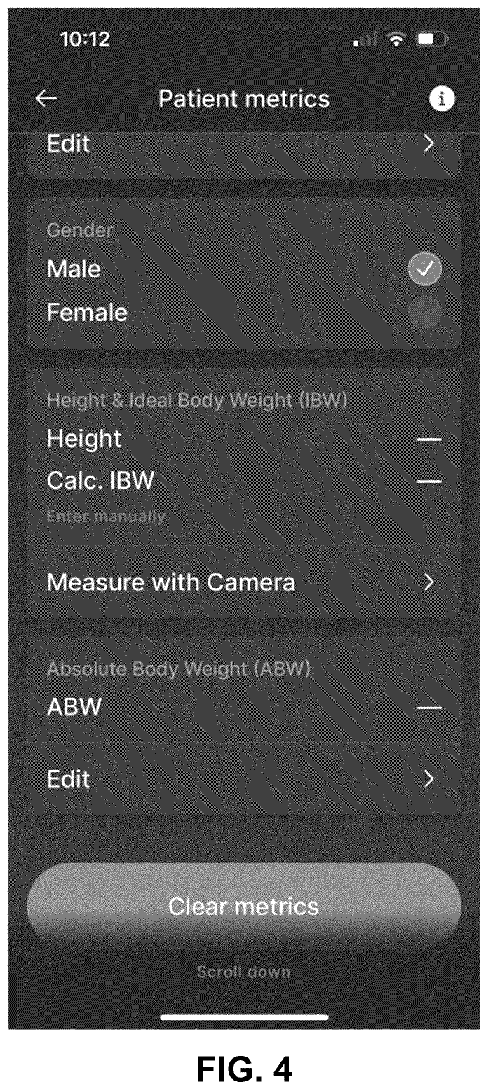

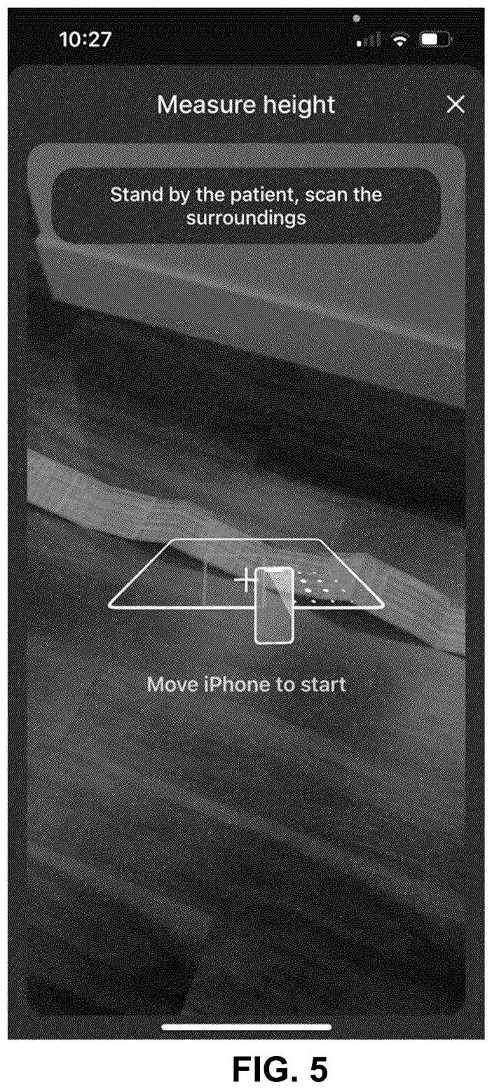

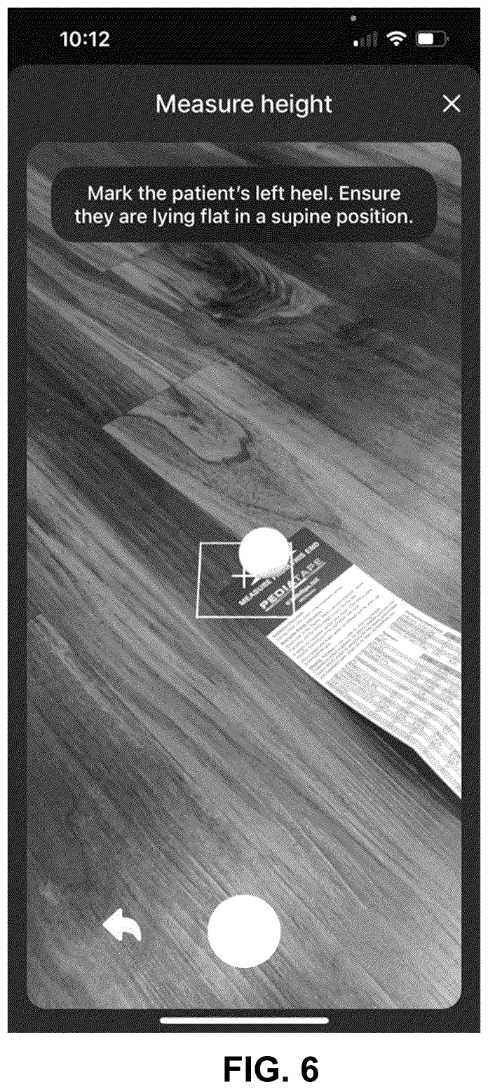

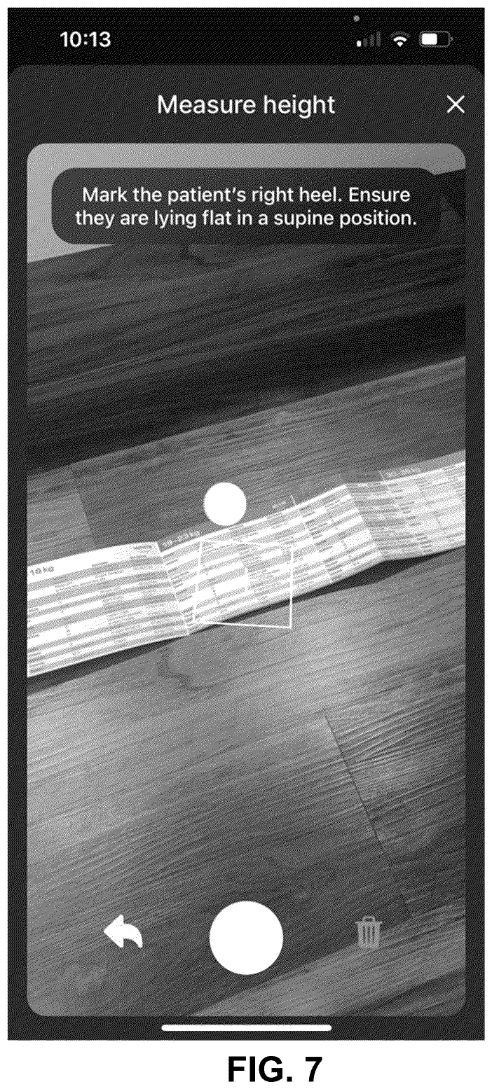

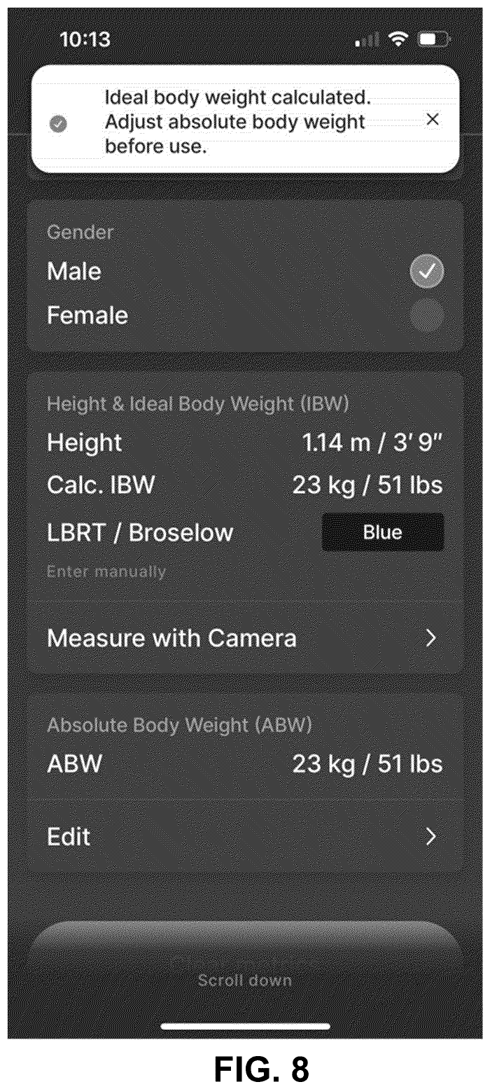

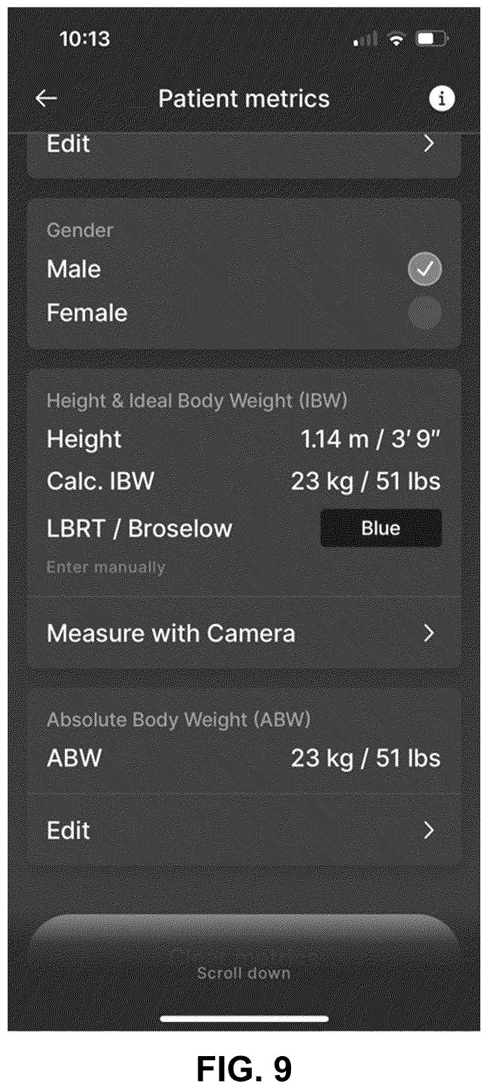

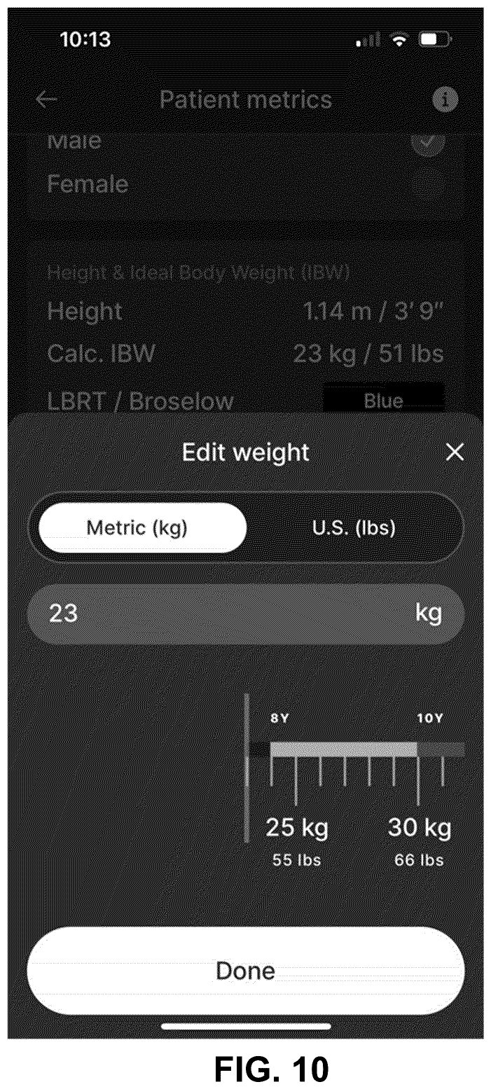

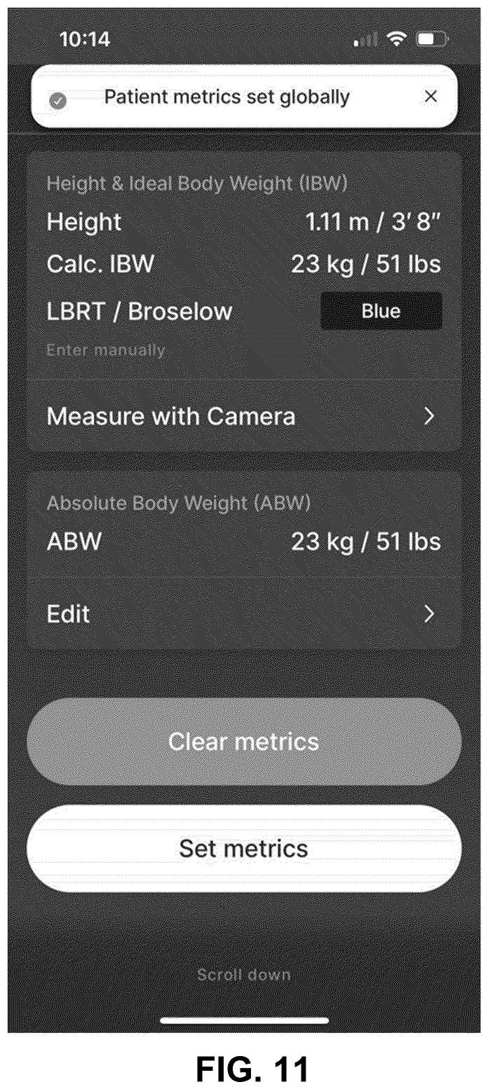

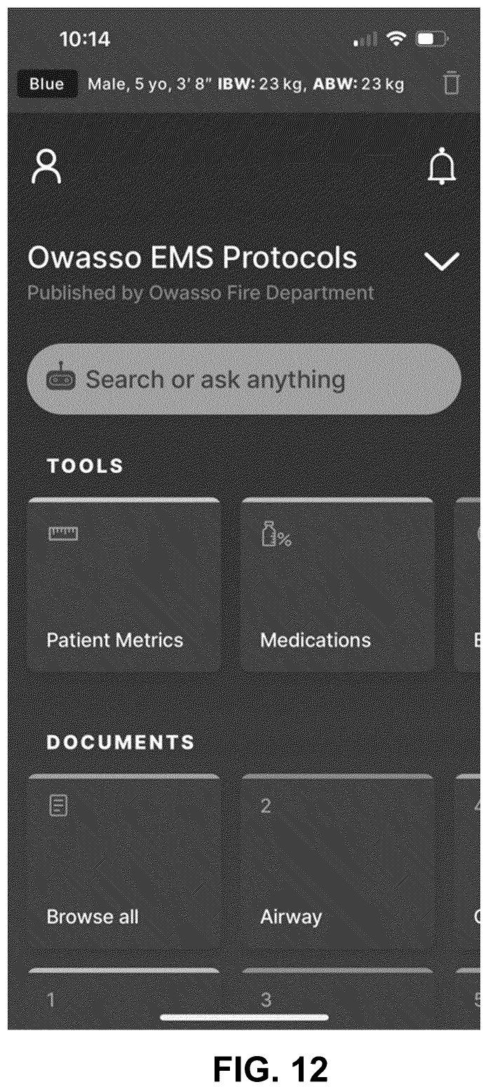

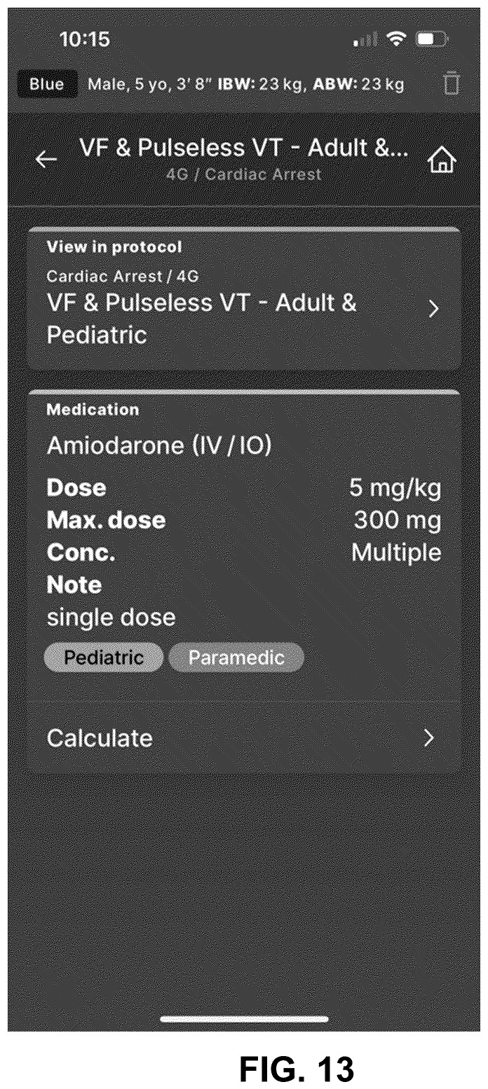

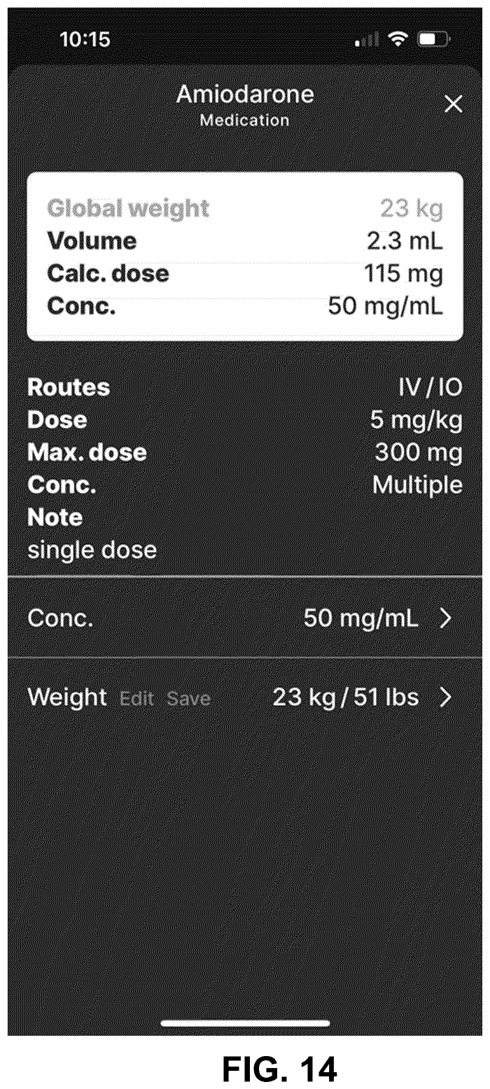

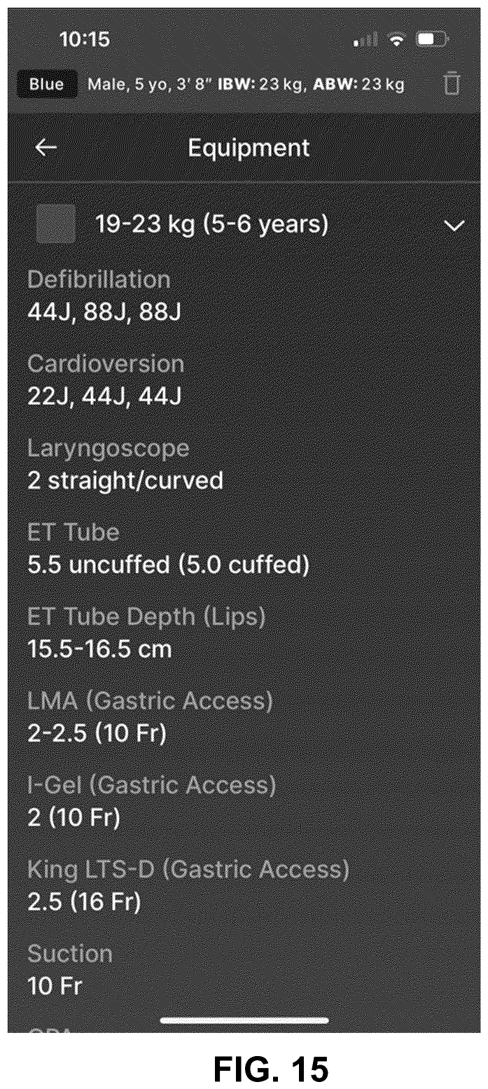

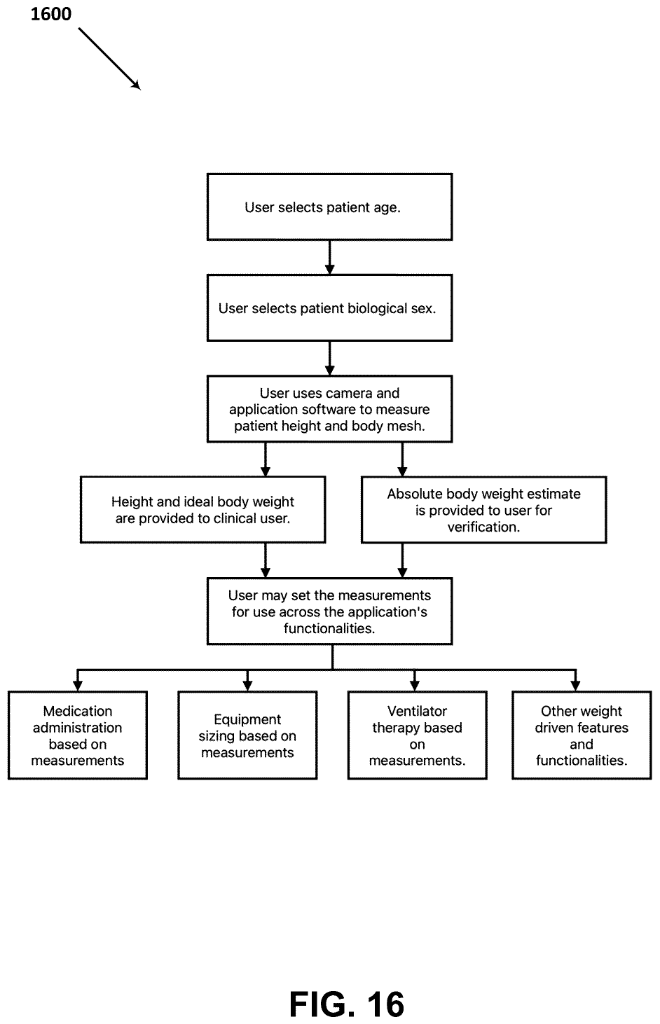

In contrast to such prior approaches, exemplary embodiments offer improved performance and capability. With reference now to FIGS. 1A through 16, and in accordance with various exemplary embodiments, an augmented reality data acquisition system 100 may comprise a patient metrics software application (for example, operative on a mobile computing device such as a smartphone or tablet) comprising an EMS application that integrates augmented reality (AR) and deep learning technologies to accurately measure patient 102 metrics and support critical decision-making by a user 101 in emergency medical situations. An exemplary mobile computing device may include processor 114, memory 116, camera 118, storage 122, communication component(s) 124, and the like. For example, exemplary embodiments may be realized on and/or comprise or include a smartphone running iOS from Apple, a smartphone running the Android operating system from Google, a tablet, and/or the like. Components and features of various exemplary embodiments are as follows:
1. Augmented Reality (AR) Measurement System: the AR component enables EMS providers to quickly and accurately measure a patient's height and calculate their ideal body weight using a smartphone or similar device. This can be done by placing virtual points at the patient's feet and head, or utilizing on-device light detection and ranging (LiDAR) technology (for example, when provided by camera/imaging component(s) 118), when equipped, without virtual point placement, generating a body mesh from scene measurements to further enhance accuracy and provide additional data points for comprehensive patient assessment, or processing purely image-based solutions to estimate patient mass given a known height, including the detection and analysis of clothing to refine mass estimation accuracy. The AR system provides a precise and reliable method for capturing patient height and deriving the ideal body weight, which is important for further calculations.
2. Deep Learning-Based Weight Estimation: Following the AR-generated measurements, an exemplary embodiment 100 employs a machine learning model, such as a deep learning-based neural network, to estimate the patient's absolute body weight. Any suitable machine learning model or models may be utilized. The model is designed to analyze and learn from a wide range of input data, including but not limited to patient demographics, biometric measurements, and other relevant data points. By leveraging a diverse and extensive dataset, the model can provide accurate weight estimations across various patient populations, regardless of age, gender, ethnicity, or other demographic factors. This approach ensures that the estimation process is adaptable and applicable to a broad spectrum of scenarios, making it a robust tool for emergency medical decision-making. Moreover, in various exemplary embodiments machine learning components may be implemented in such a way that it can process this multimodal information at different weights in different environments to provide accurate estimated absolute body weights and height. The machine learning model may be operative on the smartphone itself; alternatively, the machine learning model may be operative on external computing resources (such as server in a data center, a cloud computing resource, and/or the like) and may be in communicative connection with the smartphone to send and receive relevant data.
3. Automated Decision Support Tools: The application automatically uses the AR-measured height, calculated ideal body weight, and neural network-estimated absolute body weight to calculate critical emergency care parameters. Examples of these include:

Medication Dosages: The application determines weight-based dosing for various emergency medications, including but not limited to epinephrine, amiodarone, ketamine, atropine, fentanyl, morphine, naxalone, tranexamic acid, and the like. This is particularly important for pediatric patients, where dosages must be calculated accurately to avoid adverse effects. Exemplary embodiments replace the need for manual calculations or reliance on length-based resuscitation tapes, ensuring precise dosing based on real-time measurements.

Ventilator Settings: The application calculates appropriate ventilator settings, such as tidal volume and PEEP, based on the ideal body weight. This reduces the risk of lung injury by ensuring that ventilation strategies are tailored to the patient's specific measurements rather than estimates.

Fluid Resuscitation: The application calculates fluid resuscitation volumes based on accurate body weight for trauma patients or those in shock. This is important for managing conditions such as hypovolemic shock or severe burns, where precise fluid management can be life-saving.

Drug Administration: The application provides automated calculations for administering weight-based emergency medications, ensuring that patients receive the correct dose quickly and safely.

CPR and Defibrillation: For pediatric patients or those with specific body types, the application can assist in adjusting the force of chest compressions during CPR and selecting the appropriate defibrillation energy settings, improving the effectiveness of resuscitation efforts.

Equipment Sizing: The application assists paramedics in selecting the correct size of medical equipment, such as endotracheal tubes, laryngeal masks, and cervical collars, based on accurate patient measurements. This ensures that the chosen equipment fits properly, reducing the risk of complications during airway management, immobilization, or other procedures.

Traditionally performed using length-based resuscitation tapes for pediatric patients or estimations for adults, these calculations are now handled with a more precise, automated approach, significantly reducing the risk of error. By providing paramedics with accurate, real-time data, the application enhances the safety and efficacy of emergency interventions.

Additional Uses: While certain exemplary embodiments focus on critical emergency care parameters handled by paramedics, in various exemplary embodiments the measurements can also support a broader range of medical decisions, including radiology contrast dosing, inotropic therapy, anticoagulation management, nutritional support, and more. These additional uses highlight the application's versatility and potential in both pre-hospital and hospital settings.
4. Integration and Usability: Exemplary systems and methods introduce a seamless measure-to-action process that historically did not exist in emergency medical services. It is designed to integrate effortlessly into existing EMS workflows, transforming traditional methods into a more precise and efficient digital workflow. The intuitive user interface allows EMS providers to perform patient measurements quickly and immediately access decision-support information that is directly informed by those measurements. This eliminates the need for manual calculations or estimations, enabling providers to make rapid, data-driven decisions in critical moments. The user-friendly application requires minimal training, allowing EMS personnel to focus on patient care rather than navigating complex systems.
5. Materials and Technology: Various exemplary embodiments comprise software operative on a mobile device, such as a smartphone. It will be appreciated, however, that any suitable combination of software and hardware may be utilized to implement principles of the present disclosure. The software is developed in various programming languages and is designed to run on major mobile devices commonly used by EMS providers. It includes a neural network built using deep learning techniques integrated into the application to estimate absolute body weight.
6. Example of Use: In accordance with various exemplary embodiments, a user 101 may utilize a patient metrics software application as follows: Step 1: Activate the AR measurement tool and position the device to capture the full height of patient 102 using virtual points or LiDAR. Step 2: The AR system automatically calculates the patient's height and ideal body weight. Step 3: The deep learning model processes sensor and measurement data to estimate the patient's absolute body weight. Step 4: The application automatically uses the patient's measurements to drive any patient-measurement height—or weight-based decision support tools. Step 5: EMS providers can use patient-specific decision-support information to provide medications, ventilator therapy, and equipment sizing for their patients. In these steps, appropriate output and results may be provided within a GUI operative on the mobile computing device, allowing for rapid utilization of the information by user 101.

In accordance with various exemplary embodiments, principles of the present disclosure are utilized via certain components and process steps, as follows:

### Augmented Reality (AR) Measurement System:

Central Feature: The AR system is a core component of various exemplary embodiments. It is utilized for capturing patient height and calculating ideal body weight, which are foundational to the subsequent decision-support calculations. EMS providers activate the AR tool and use it to measure the patient's height. This step is central as it provides the initial data utilized for further processing by the neural network and decision-support tools.

### Deep Learning-Based Neural Network:

Central Feature: The deep learning-based neural network is likewise central to various exemplary embodiments. It processes the AR-generated measurements to estimate the patient's absolute body weight. This neural network can be trained on a diverse dataset and is utilized for providing accurate weight estimations across different patient populations. The neural network processes the height and ideal body weight data to estimate the absolute body weight. This step is central, as it directly influences the accuracy of the automated decision-support tools. Moreover, as exemplary embodiments are utilized, weight estimations for patients can be improved in robustness, speed, and accuracy as additional patient data is collected into the system.

### Automated Decision Support Tools:

Central Feature: These tools use the data generated by the AR system and neural network to calculate critical emergency care parameters such as medication dosages, ventilator settings, fluid resuscitation volumes, and equipment sizing. The application automatically calculates and displays these parameters based on the patient's measurements. This step ensures that EMS providers receive real-time, accurate information to make informed decisions during emergency care.

Optional Features, Parts, and Steps: Various exemplary embodiments utilize additional and/or differing components from one another. For example:

LiDAR Technology Integration: the use of on-device LiDAR technology for measurement is optional, but is included in various exemplary embodiments. While it enhances the accuracy and ease of capturing patient height, the AR tool can also function with virtual point placement, making LiDAR an optional enhancement.

Optional Step: If LiDAR technology is available on the device, the EMS provider can choose to use it for a hands-free measurement process. However, this step can be bypassed if LiDAR is absent or the provider prefers virtual point placement.

### Additional Medical Decision-Support Tools:

Optional Feature: In various exemplary embodiments, the application includes additional decision-support tools, such as those for radiology contrast dosing, inotropic therapy, or anticoagulation management. While these tools increase the application's utility, they are not essential for its core functionality.

Optional Step: EMS providers may choose to use these additional tools depending on the patient's specific needs and situation. These steps are optional and dependent on the specific scenario and available resources.

In accordance with various exemplary embodiments, a patient metrics software application has been developed, incorporating ARKit for iOS devices, ARCore for Android devices, and a deep learning-based neural network created using PyTorch. This embodiment successfully implemented the ability to capture height measurements using AR and calculate ideal body weight. A neural network has been utlized to estimate absolute body weight, showing promising results. The ongoing development includes experiments to refine the neural network's accuracy. This involves testing with additional datasets, optimizing the model's architecture, and improving its ability to generalize across different patient demographics. These revisions are desirable for ensuring that the neural network can reliably perform in real-world emergency medical scenarios.

LiDAR vs. Virtual Point Placement:

In accordance with various exemplary embodiments, testing was conducted to compare the accuracy of height measurements using LiDAR technology versus virtual point placement. The results indicated that while LiDAR provides higher accuracy, the virtual point placement method is sufficiently accurate and reliable for most emergency care scenarios. Accordingly, exemplary systems and methods may utilize either approach.

An exemplary embodiment includes using on-device LiDAR technology for height measurement combined with a deep learning-based neural network for weight estimation. This configuration provides a high degree of accuracy and ease of use while reducing the computational and communicative burden on the mobile device, making it ideal for emergency care settings where speed and precision are critical.

Certain embodiments incorporate all the core features—AR measurement (using ARKit for iOS and ARCore for Android), deep learning-based weight estimation, and automated decision support—into a single, user-friendly application that integrates seamlessly into EMS workflows. These embodiments are designed to be highly adaptable, with optional features like additional decision-support tools and the use of LiDAR technology depending on the available equipment and specific needs of the EMS provider.

With continued reference to FIGS. 1A-16, in various exemplary embodiments a patient metrics software application comprises various sub-systems and capabilities.
1. Augmented Reality (AR) Measurement System: The AR measurement system is designed to accurately capture patient height and calculate ideal body weight, forming the foundation for subsequent calculations. This system can be implemented using various AR platforms, including ARKit for iOS and ARCore for Android, but is not limited to these technologies. Other embodiments may utilize other AR platforms or custom-developed AR solutions.

In various exemplary embodiments, the AR system uses the camera and sensors of a mobile device, including accelerometers, gyroscopes, and/or LiDAR, to generate accurate measurements. For example, the AR system may generate a body mesh from scene measurements to further enhance accuracy and provide additional data points for comprehensive patient assessment; alternatively, the AR system may implement a purely image-based solution to estimate patient mass given a known height, including the detection and analysis of clothing to refine mass estimation accuracy. While some embodiments use mobile devices like smartphones and tablets, the system may be utilized with other portable devices, wearable technology, or integrated into specialized EMS equipment.

The AR system is desirable utilized in emergency medical situations where quick, accurate patient measurements are critical. However, it can be applied in other medical applications, such as in-hospital patient assessment, home healthcare, or remote consultations.

Alternative implementations utilize different sensor technologies, such as infrared cameras or ultrasound, to capture patient measurements. The AR system may also be used for full-body scanning for more comprehensive patient assessments. Exemplary embodiments generate a precise and reliable measurement of patient height and ideal body weight, providing a core input for the subsequent decision-support processes.
2. Deep Learning-Based Neural Network: In various exemplary embodiments, the neural network is designed to estimate absolute body weight based on AR-generated measurements and additional input data. While some embodiments utilize a deep learning-based neural network developed with PyTorch, other machine learning frameworks, such as TensorFlow or custom-developed algorithms, may also be employed.

In some exemplary embodiments, the neural network is initially developed in a high-level programming language like Python using machine learning frameworks and is later compiled into C for optimized performance. The network architecture can vary, with alternatives including convolutional neural networks (CNNs), recurrent neural networks (RNNs), or hybrid models that combine multiple approaches.

The neural network's primary application is in emergency medical services, where it is used to estimate patient body weight accurately and rapidly, which is desirable for dosing, equipment sizing, and other critical decisions. The neural network may also be used in other healthcare settings, such as outpatient clinics, rehabilitation centers, or sports medicine.

In some exemplary embodiments, the neural network is trained on a broader range of datasets to improve accuracy across diverse populations. Additionally, alternative models, such as decision trees, support vector machines (SVMs), or ensemble methods, may be utilized depending on the application's specific requirements. The neural network provides a highly accurate estimation of patient body weight, enabling more precise medical interventions and improving patient outcomes.
3. Automated Decision Support Tools: In various exemplary embodiments, these tools utilize the measurements and estimations provided by the AR system and neural network to calculate and recommend important emergency care parameters. The tools cover various calculations, from medication dosages to ventilator settings and equipment sizing. The decision support tools are integrated into the EMS application software and are designed to be highly adaptable. As medical protocols evolve, additional tools can be included or existing ones modified. These tools are primarily used by EMS providers in the field, but they are also suitable for use in hospitals, clinics, or even home healthcare settings. They may also be used in training environments to simulate real-world scenarios for EMS personnel. The tools may be revised or expanded to include additional parameters, such as predictive analytics for patient outcomes, or integrated with other healthcare data systems to provide a more comprehensive view of the patient's condition. Moreover, certain embodiments focus on specific use cases, such as pediatric care or geriatric care, with customized decision-support algorithms. These approaches enable the delivery of accurate, real-time decision-support information that enhances the safety, efficacy, and speed of emergency medical interventions.

In various exemplary embodiments, the patient metrics software application is designed for seamless integration into existing EMS workflows, emphasizing ease of use and minimal training requirements. The system may be integrated with other healthcare systems, such as electronic health records (EHRs) or telemedicine platforms. The user interface is developed to be intuitive and user-friendly, and may comprise components including touchscreens, voice commands, or gesture-based controls. Exemplary embodiments are platform-agnostic, ensuring compatibility with various mobile devices and operating systems.

In various exemplary embodiments, the patient metrics software application is configured with additional usability features, such as customizable user interfaces, multilingual support, or accessibility options for providers with disabilities. The application may also interface with medical devices through Bluetooth or other means of electronic transmission to drive medication infusions, ventilators, and other device-delivered therapies. In this manner, exemplary embodiments provide a highly efficient, user-friendly system that integrates seamlessly into the EMS workflow, enabling providers to focus on patient care rather than navigating complex systems.

Exemplary embodiments are implemented using various programming languages and technologies that ensure compatibility with major mobile device platforms. Moreover, the neural network can be developed with common deep learning frameworks and compiled to lower-level languages, such as c, for cross-device compatibility. In various exemplary embodiments, the application is designed to run on iOS and Android devices, leveraging ARKit and ARCore for AR capabilities. The neural network's construction in C ensures that it operates efficiently even in resource-constrained environments, such as on mobile devices used in the field. While a primary use of exemplary systems and methods are in EMS, the materials and technology choices in exemplary embodiments allow for use in other industries where rapid, accurate measurements and decision support are desirable, such as military medicine, sports science, or remote monitoring. It will be appreciated that alternative programming languages or machine learning frameworks could be used, depending on future technological advancements or specific application needs, and principles of the present disclosure contemplate all such approaches. Exemplary embodiments may also be used in augmented reality glasses or other wearable devices. In this manner, exemplary embodiments deliver a robust, efficient, and adaptable application that meets the rigorous demands of emergency medical services.

As compared to prior approaches, exemplary embodiments uniquely combine augmented reality (AR) and deep learning technologies to provide accurate and real-time patient measurements. The AR system captures patient height and calculates body weight, while a deep learning-based neural network estimates absolute body weight. This seamless integration of AR and AI-driven analysis in a mobile application tailored for EMS use is a novel approach that addresses addressing the critical need for accurate and rapid patient assessment in emergencies to perform these measurements and calculations on standard mobile devices without the need for addition without the need for additional hardware existing solutions.

Moreover, exemplary embodiments offer real-time, automated decision support based on the AR-measured height and neural network-estimated weight. It calculates essential emergency care parameters, such as medication dosages, ventilator settings, and equipment sizing, all within a single workflow. The automated measure-to-action process, which eliminates the need for manual calculations or reliance on rough estimations, significantly reduces the risk of human error. This process is particularly advantageous in high-pressure, time-sensitive environments like emergency medical services, making it an important differentiator from traditional methods.

Additionally, exemplary embodiments are designed to operate on widely available mobile devices using ARKit for iOS and ARCore for Android, ensuring broad accessibility and ease of deployment across different EMS organizations. Unlike specialized equipment or proprietary systems that require significant investment and training, this platform-agnostic approach allows for rapid adoption and integration into existing workflows, enhancing its practicality and utility in the field.

Yet further, in exemplary embodiments the deep learning model is trained on a diverse dataset to ensure accurate weight estimation across various patient demographics, including different ages, genders, and ethnic backgrounds. This focus on adaptability ensures that the application provides reliable results for a wide range of patients. This is a significant improvement over traditional methods that may be less accurate or less applicable to certain populations.

Still other benefits compared to prior approaches exist, including that the user interface is designed to be intuitive and user-friendly, requiring minimal training for EMS providers. The application seamlessly integrates into existing EMS workflows, ensuring that providers can quickly and efficiently use the tool without disruption. The ease of use and minimal training requirements distinguish exemplary embodiments from more complex systems that might require extensive onboarding or disrupt existing workflows. The focus on usability ensures that the application enhances, rather than complicates, the EMS provider's work.

Based on use of certain exemplary embodiments, it has been determined that the level of accuracy in capturing patient height, even without the use of LiDAR, exceeds expectations. Preliminary results suggest that the exemplary embodiments will perform well under various conditions, providing quick and accurate measurements. Achieving high accuracy across diverse conditions is a surprising validation of its effectiveness. This result would suggest that exemplary embodiments may be reliably used in emergency scenarios without the need for additional hardware, which was not initially apparent.

In addition to the foregoing disclosed embodiments, additional embodiments may extend the capabilities of and/or applications of the system. For example, certain embodiments may be extended to integrate directly with various medical devices that require accurate patient measurements. For example, the AR and neural network-generated data could be transmitted in real-time to ventilators, infusion pumps, or defibrillators to automatically adjust settings based on the patient's specific measurements. The data can also be transmitted to and/or displayed on a GUI of other devices, such as a screen in an ambulance, a smart stretcher or patient transport bed, and/or the like. This integration enables a seamless flow of data from the patient assessment phase to the care administration, reducing the need for manual input and minimizing the risk of errors. It also allows medical devices to operate more efficiently and accurately, tailoring their operation to the patient's precise needs.

Moreover, certain embodiments may be extended to integrate with Electronic Health Records (EHR) systems, allowing patient measurements and related decision-support data to be automatically uploaded and stored as part of the patient's medical record. This ensures that all relevant patient data is captured and easily accessible to healthcare providers across different settings, from the field to the hospital. It also facilitates better continuity of care, as the data gathered in emergency situations is available for review and further action by other medical professionals.

Yet further, exemplary embodiments may include additional decision support tools tailored to specific medical conditions. For instance, the system may provide specialized guidance for managing conditions such as sepsis, trauma, or stroke, where patient measurements are critical for determining treatment protocols. By offering condition-specific decision support, the system becomes an even more valuable tool for EMS providers, enabling them to make more informed decisions in complex cases. This can improve patient outcomes in a wider range of emergency scenarios.

Additionally, exemplary embodiments may support telemedicine applications, allowing EMS providers to share real-time patient measurements and decision support data with remote physicians or specialists. This extension enables more collaborative care, with remote experts able to provide guidance based on accurate, up-to-date patient data. It also facilitates remote patient management when direct access to a physician is impossible, such as in rural or underserved areas.

Still further, exemplary embodiments may include advanced analytics and predictive modeling tools that analyze patient data over time to forecast potential complications or outcomes. These tools provide EMS providers with additional insights, helping them to anticipate and address issues before they become critical. This integration enhances the application's ability to support proactive care, allowing for earlier interventions based on predicted risks. It also contributes to more personalized treatment plans tailored to each patient's unique needs and risks.

Further still, exemplary embodiments may utilize AR technology incorporated into additional devices used by EMS clinicians, such as smart glasses, heads-up displays, or monitors installed in ambulances and emergency rooms. These devices can provide real-time overlays of patient measurements, guidance for procedures, or navigation during transport. By integrating AR into a broader range of EMS tools and workspaces, clinicians may access critical information hands-free and in real time, enhancing situational awareness and decision-making. This leads to more efficient workflows, reduced cognitive load, and better patient outcomes, particularly in high-stress environments where quick access to information is crucial.

In a number of embodiments, the techniques described herein can solve a technical problem that arises only within the realm of computers and computer networks, as augmented reality techniques do not exist outside the realm of computers and computer networks.

The present system, or any part(s) or function(s) thereof, may be implemented using hardware, software, or a combination thereof and may be implemented in one or more computer systems or other processing systems. However, the manipulations performed by embodiments were often referred to in terms, such as matching or selecting, which are commonly associated with mental operations performed by a human operator. No such capability of a human operator is necessary, or desirable in most cases, in any of the operations described herein. Rather, the operations may be machine operations or any of the operations may be conducted or enhanced by artificial intelligence (AI) or machine learning. Artificial intelligence may refer generally to the study of agents (e.g., machines, computer-based systems, etc.) that perceive the world around them, form plans, and make decisions to achieve their goals. Foundations of AI include mathematics, logic, philosophy, probability, linguistics, neuroscience, and decision theory. Many fields fall under the umbrella of AI, such as computer vision, robotics, machine learning, and natural language processing. Useful machines for performing the various embodiments include general purpose digital computers or similar devices.

Further, illustrations of the process flows and the descriptions thereof may make reference to user WINDOWS® applications, webpages, websites, web forms, prompts, etc. Practitioners will appreciate that the illustrated steps described herein may comprise any number of configurations including the use of WINDOWS® applications, webpages, web forms, popup WINDOWS® applications, prompts, and the like. It should be further appreciated that the multiple steps as illustrated and described may be combined into single webpages and/or WINDOWS® applications but have been expanded for the sake of simplicity. In other cases, steps illustrated and described as single process steps may be separated into multiple webpages and/or WINDOWS® applications but have been combined for simplicity.

In various embodiments, components, modules, and/or engines of system 100 may be implemented as micro-applications, micro-apps, micro-services, or the like. Micro-apps are typically deployed in the context of a mobile operating system, including for example, a WINDOWS® mobile operating system, an ANDROID® operating system, an APPLE® iOS operating system, or any other operating system that may be readily apparent to one skilled in the art. The micro-app may be configured to leverage the resources of the larger operating system and associated hardware via a set of predetermined rules which govern the operations of various operating systems and hardware resources. For example, where a micro-app desires to communicate with a device or network other than the mobile device or mobile operating system, the micro-app may leverage the communication protocol of the operating system and associated device hardware under the predetermined rules of the mobile operating system. Moreover, where the micro-app desires an input from a user, the micro-app may be configured to request a response from the operating system which monitors various hardware components and then communicates a detected input from the hardware to the micro-app.

The systems, computers, computer-based systems, and the like disclosed herein may provide a suitable website or other internet-based graphical user interface which is accessible by users. Practitioners will appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

Any of the communications, inputs, storage, databases or displays discussed herein may be facilitated through a website having web pages. The term “web page” as it is used herein is not meant to limit the type of documents and applications that might be used to interact with the user. For example, a typical website might include, in addition to standard HTML documents, various forms, JAVA® applets, JAVASCRIPT® programs, active server pages (ASP), common gateway interface scripts (CGI), extensible markup language (XML), dynamic HTML, cascading style sheets (CSS), AJAX (Asynchronous JAVASCRIPT and XML) programs, helper applications, plug-ins, and the like. A server may include a web service that receives a request from a web server, the request including a URL and an IP address. The web server retrieves the appropriate web pages and sends the data or applications for the web pages to the IP address. Web services are applications that are capable of interacting with other applications over a communications means, such as the internet. Web services are typically based on standards or protocols such as XML, SOAP, AJAX, WSDL and UDDI. Web services methods are well known in the art, and are covered in many standard texts. As a further example, representational state transfer (REST), or RESTful, web services may provide one way of enabling interoperability between applications. In various embodiments, any communication discussed herein may be accomplished via the internet or an intranet. Communications may be completed using any suitable protocol, such as, for example, IPv4 (base 10), IPV6 (HMAC), and/or any other suitable or desired communications protocol.

In one embodiment, MICROSOFT® company's Internet Information Services (IIS), Transaction Server (MTS) service, and an SQL SERVER® database, are used in conjunction with MICROSOFT® operating systems, WINDOWS NT® web server software, SQL SERVER database, and MICROSOFT® Commerce Server. Additionally, components such as ACCESS® software, SQL SERVER® database, ORACLE® software, SYBASE® software, INFORMIX® software, MYSQL® software, INTERBASE® software, etc., may be used to provide an Active Data Object (ADO) compliant database management system. In one embodiment, the APACHE® web server is used in conjunction with a LINUX® operating system, a MYSQL® database, and PERL®, PHP, Ruby, and/or PYTHON® programming languages.

In various embodiments, the server may include application servers (e.g., WEBSPHERE®. WEBLOGIC®, JBOSS®, POSTGRES PLUS ADVANCED SERVER®, etc.). In various embodiments, the server may include web servers (e.g., Apache, IIS, GOOGLE® Web Server, SUN JAVA® System Web Server, JAVA® Virtual Machine running on LINUX® or WINDOWS® operating systems). In various embodiments, service solutions may also include IaaS environments, PaaS environments, and/or the like.

Users, systems, computer-based systems, or the like may communicate with the server via a web client. The web client includes any device or software which communicates via any network such as, for example any device or software discussed herein. The web client may include internet browsing software installed within a computing unit or system to conduct online transactions and/or communications. These computing units or systems may take the form of a computer or set of computers, although other types of computing units or systems may be used, including personal computers, laptops, notebooks, tablets, smart phones, cellular phones, personal digital assistants, servers, pooled servers, mainframe computers, distributed computing clusters, kiosks, terminals, point of sale (POS) devices or terminals, televisions, or any other device capable of receiving data over a network. The web client may include an operating system (e.g., WINDOWS®, WINDOWS MOBILE® operating systems, UNIX® operating system, LINUX® operating systems, APPLE® OS® operating systems, etc.) as well as various conventional support software and drivers typically associated with computers. The web-client may also run MICROSOFT® INTERNET EDGE® software, MOZILLA FIREFOX® software, GOOGLE® CHROME® software, APPLE® SAFARI® software, or any other of the myriad software packages available for browsing the internet.

As those skilled in the art will appreciate, the web client may or may not be in direct contact with the server (e.g., application server, web server, etc., as discussed herein). For example, the web client may access the services of the server through another server and/or hardware component, which may have a direct or indirect connection to an internet server. For example, the web client may communicate with the server via a load balancer. In various embodiments, web client access is through a network or the internet through a commercially available web-browser software package. In that regard, the web client may be in a home or business environment with access to the network or the internet. The web client may implement security protocols such as Secure Sockets Layer (SSL) and Transport Layer Security (TLS). A web client may implement several application layer protocols including HTTP, HTTPS, FTP, and SFTP.

Any databases discussed herein may include relational, hierarchical, graphical, blockchain, object-oriented structure, and/or any other database configurations. In various embodiments, any database may also include a no-SQL database, a key-value database, an in-memory database, a GPU database, and/or the like. Any database may also include a flat file structure wherein data may be stored in a single file in the form of rows and columns, with no structure for indexing and no structural relationships between records. For example, a flat file structure may include a delimited text file, a CSV (comma-separated values) file, and/or any other suitable flat file structure. Common database products that may be used to implement the databases include DB2® by IBM® (Armonk, NY), various database products available from ORACLE® Corporation (Redwood Shores, CA), MICROSOFT ACCESS® or MICROSOFT SQL SERVER® by MICROSOFT® Corporation (Redmond, Washington), MYSQL® by MySQL AB (Uppsala, Sweden), MONGODB®, Redis, Apache Cassandra®, HBASE® by APACHE®, MapR-DB by the MAPR® corporation, or any other suitable database product. Moreover, any database may be organized in any suitable manner, for example, as data tables or lookup tables. Each record may be a single file, a series of files, a linked series of data fields, or any other data structure.

Any database discussed herein may comprise a distributed ledger maintained by a plurality of computing devices (e.g., nodes) over a peer-to-peer network. Each computing device maintains a copy and/or partial copy of the distributed ledger and communicates with one or more other computing devices in the network to validate and write data to the distributed ledger. The distributed ledger may use features and functionality of blockchain technology, including, for example, consensus-based validation, immutability, and cryptographically chained blocks of data. The blockchain may comprise a ledger of interconnected blocks containing data. The blockchain may provide enhanced security because each block may hold individual transactions and the results of any blockchain executables. Each block may link to the previous block and may include a timestamp. Blocks may be linked because each block may include the hash of the prior block in the blockchain. The linked blocks form a chain, with only one successor block allowed to link to one other predecessor block for a single chain. Forks may be possible where divergent chains are established from a previously uniform blockchain, though typically only one of the divergent chains will be maintained as the consensus chain. In various embodiments, the blockchain may implement smart contracts that enforce data workflows in a decentralized manner. The system may also include applications deployed on user devices such as, for example, computers, tablets, smartphones, Internet of Things devices (“IoT” devices), etc. The applications may communicate with the blockchain (e.g., directly or via a blockchain node) to transmit and retrieve data. In various embodiments, a governing organization or consortium may control access to data stored on the blockchain. Registration with the managing organization(s) may enable participation in the blockchain network.

The system and method may be described herein in terms of functional block components, screen shots, optional selections, and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C #, JAVA®, JAVASCRIPT®, JAVASCRIPT® Object Notation (JSON), VBScript, Macromedia COLD FUSION, COBOL, MICROSOFT® company's Active Server Pages, assembly, PERL®, PHP, awk, PYTHON®, Visual Basic, SQL Stored Procedures, PL/SQL, any UNIX® shell script, and extensible markup language (XML) with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JAVASCRIPT®, VBScript, or the like. Cryptography and network security methods are well known in the art and are covered in many standard texts.

In various embodiments, the software elements of the system may also be implemented using a JavaScript® run-time environment configured to execute JavaScript® code outside of a web browser. For example, the software elements of the system may be implemented using NODE. JS® components. NODE. JS® programs may implement several modules to handle various core functionalities. For example, a package management module, such as NPM®, may be implemented as an open-source library to aid in organizing the installation and management of third-party NODE.JS® programs. NODE.JS® programs may also implement a process manager such as, for example, Parallel Multithreaded Machine (“PM2”); a resource and performance monitoring tool such as, for example, Node Application Metrics (“appmetrics”); a library module for building user interfaces, and/or any other suitable and/or desired module.

As will be appreciated by one of ordinary skill in the art, the system may be embodied as a customization of an existing system, an add-on product, a processing apparatus executing upgraded software, a stand-alone system, a distributed system, a method, a data processing system, a device for data processing, and/or a computer program product. Accordingly, any portion of the system or a module may take the form of a processing apparatus executing code, an internet-based embodiment, an entirely hardware embodiment, or an embodiment combining aspects of the internet, software, and hardware. Furthermore, the system may take the form of a computer program product on a computer-readable storage medium having computer-readable program code means embodied in the storage medium. Any suitable computer-readable storage medium may be utilized, including hard disks, CD-ROM, SONY BLU-RAY DISC®, optical storage devices, magnetic storage devices, and/or the like.

While the principles of this disclosure have been shown in various embodiments, many modifications of structure, arrangements, proportions, the elements, materials and components, used in practice, which are particularly adapted for a specific environment and operating requirements may be used without departing from the principles and scope of this disclosure. These and other changes or modifications are intended to be included within the scope of the present disclosure and may be expressed in the following claims.

The present disclosure has been described with reference to various embodiments. However, one of ordinary skill in the art appreciates that various modifications and changes can be made without departing from the scope of the present disclosure. Accordingly, the specification is to be regarded in an illustrative rather than a restrictive sense, and all such modifications are intended to be included within the scope of the present disclosure. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element of any or all the claims.

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. When language similar to “at least one of A, B, or C” or “at least one of A, B, and C” is used in the claims or specification, the phrase is intended to mean any of the following: (1) at least one of A; (2) at least one of B; (3) at least one of C; (4) at least one of A and at least one of B; (5) at least one of B and at least one of C; (6) at least one of A and at least one of C; or (7) at least one of A, at least one of B, and at least one of C.

What is claimed is:

1. A computerized method for determining and utilizing patient metrics in an emergency medicine context via use of augmented reality (AR) and machine learning (ML), the method comprising:
obtaining, via a camera system of a mobile device, information regarding the height of a patient, wherein the obtaining utilizes augmented reality capabilities of the camera system;
determining, by a machine learning system operative on the mobile device, an estimated weight of the patient, and
recommending, by a decision support system operative on the mobile device, treatment parameters for the patient based on the determining.

2. The method of claim 1, wherein the machine learning utilizes a deep learning based neural network.

3. The method of claim 1, wherein the camera system comprises light detection and ranging (LiDAR) capability, and wherein the obtaining information regarding the height of a patient utilizes the camera system.

4. The method of claim 1, wherein the camera system lacks LiDAR capability.

5. The method of claim 1, wherein the treatment comprises administering a medication, and wherein the treatment parameters comprise dosage information based on the weight of the patient.

6. The method of claim 1, wherein the treatment comprises utilizing a medical device, and wherein the treatment parameters comprise sizing information for the medical device based on the weight of the patient.

7. The method of claim 1, wherein the treatment comprises ventilating the patient, and wherein the treatment parameters comprise ventilator configuration information based on the weight of the patient.

8. The method of claim 7, wherein the ventilator configuration information comprises tidal volume and positive end-expiratory pressure (PEEP).

9. The method of claim 1, wherein determining the estimated weight of the patient comprises generating a mesh representative of the body of the patient.

10. The method of claim 1, wherein the recommending comprises displaying, on a graphical user interface of the mobile device, treatment parameters comprising dosage information based on the weight of the patient.

11. The method of claim 10, further comprising administering, to the patient, medication corresponding to the dosage information.

12. The method of claim 11, further comprising transmitting, to a patient treatment database and by the mobile device, a record of the administered medication.

13. The method of claim 1, further comprising inputting, to a patient treatment database and by the mobile device, a record of the administered medication.
